(19) United States
(12) Patent Application Publication
(10) Pub. No.: US 2018/0098744 A1
(43) Pub. Date: Apr. 12, 2018

(54) METHOD FOR DETERMINING AN X-RAY IMAGE DATASET AND X-RAY SYSTEM

(71) Applicant: Sebastain Bauer, Erlangen (DE)

(72) Inventor: Sebastain Bauer, Erlangen (DE)

(21) Appl. No.: 15/730,067

(22) Filed: Oct. 11, 2017

(30) Foreign Application Priority Data
Oct. 12, 2016 (DE) 102016219817.3

(51) Int. Cl. A61B 6/00; A61B 6/03

(52) U.S. Cl. CPC A61B 6/4464; A61B 6/5258; A61B 6/032

(57) ABSTRACT

A method includes recording projection images showing a target area from different projection directions along a recording trajectory. A first part of the projection images collimated onto the target region and a second part of the projection images showing the examination object non-truncated in at least one direction are recorded, so that from the second part of the projection images, at least one item of truncation information that is used in the reconstruction of the X-ray image dataset for reducing truncation artifacts is derived. The first part of the X-ray images is recorded with a first recording arrangement, and the second part of the X-ray images is recorded with a second of the recording arrangements. In each case, the collimation remains constant during the recording process. The second part of the projection images includes fewer projection images than the first part.

This application claims the benefit of DE 10 2016 219 817.3, filed on Oct. 12, 2016, which is hereby incorporated by reference in its entirety.

### BACKGROUND

The present embodiments relate to determining an X-ray image dataset of a target area of an examination object.

In X-ray imaging, it has long been known to determine higher-dimensional image datasets from lower-dimensional projection images. Therein, for example, two-dimensional projection images are recorded at different projection directions along a recording trajectory (e.g., a circular path), in order then to be able to determine a three-dimensional image dataset from the two-dimensional projection images by reconstruction methods such as iterative reconstruction and/or filtered back projection. It is also known to use one-dimensional projection images for determining two-dimensional sectional images. In order to be able to record such X-ray image datasets, it is known to use computed tomography X-ray systems in a targeted way, in which, for example, the recording arrangement including an X-ray radiator and an X-ray detector are guided within a gantry round the patient. Since, however, higher-dimensional (e.g., three-dimensional) image datasets have also proved to be useful in relation to procedures or interventions, it has been proposed to determine computed tomography-like images with other X-ray systems too (e.g., X-ray systems with a C-arm on which an X-ray radiator and an X-ray detector are fastened opposing one another). With a suitable movability of the C-arm (e.g., a pivotability), recording trajectories may be realized to scan projection images that permit the reconstruction of a three-dimensional image dataset.

Computed tomography, whether with a dedicated computed tomography X-ray system or, for example, as C-arm computed tomography involves, like every X-ray imaging method, the use of ionizing radiation. Therefore, many methods are directed to reducing the quantity of ionizing radiation that is used for a scan, which is advantageous both for the patient and for the personnel. It has, for example, been proposed thereby to restrict the imaging by a collimator to the actual target area of interest (e.g., to a volume of interest). In this way, the X-ray dose may be significantly reduced since, due to the collimation, X-rays are blocked in regions outside a pre-determined target area during the data acquisition. This has the further advantage that the scattered radiation is significantly reduced, so that an improved diagnostic image quality is produced. However, collimated projection image data is subject to truncation, which provides that in the plane of the recording trajectory, the examination object is not fully imaged. A restriction of this type is not compatible with the typically used reconstruction algorithms. If these are used, however, then truncation artifacts may occur.

In order to reduce these truncation artifacts, a large number of proposals have already been made in the prior art. It is known, for example, to estimate truncated projection image data and to add the estimated truncated projection image data back to the projection images (e.g., detruncation). A first approach thereto is based on the use of anthropomorphic heuristics, for example, the assumption that a patient to be scanned may be represented as a water cylinder. Examples of such processes are given in the articles by J. Hsieh et al., “A novel reconstruction algorithm to extend the CT scan field-of-view,” Medical Physics, Volume 31, No. 9, pages 2385 to 2391, 2004 and by K. Sourbelle et al., “Reconstruction from truncated projections in CT using adaptive detruncation,” European Radiology, Volume 15, No. 5, pages 1008 to 1014, 2005.

Disadvantages of such approaches are that extremely simple assumptions that result in reconstruction artifacts (e.g., remaining cupping artifacts and false HU values) are made. Another approach for estimating truncated projection images (e.g., detruncating) uses a patient-specific non-truncated pre-CT-scan from which a three-dimensional pre-image dataset is produced. From this, by forward projection, projection images of a non-truncated type may again be obtained. Projection images of the non-truncated type serve to complement the truncated projection images (see, e.g., the article by D. Kolditz et al., “Volume-of-interest (VOI) imaging in C-arm flat-detector CT for high image quality at reduced dose,” Medical Physics 37 (6), pages 2719 to 2730, 2010). This approach is disadvantageous in that a non-truncated pre-CT scan is to be present, and consequently, the patient is exposed to further ionizing radiation. The patient is not to be moved between the recording of the pre-image dataset, or a possibly complex 2D-3D registration is to be carried out to remove movement artifacts.

Another approach is the use of reconstruction algorithms that prove to be robust with regard to the truncation (see, e.g., the article by F. Dennerlein and A. Maier, “Approximate truncation robust computed tomography—ATRACT,” Physics in Medicine and Biology, Volume 58, pages 6133 to 6148, 2013). With such algorithms also, however, persisting reconstruction artifacts occur, and there is an offset/scaling problem.

Physically present semi-transparent X-ray filters for the region to be cut off (e.g., the region to be truncated) are to be provided, so that the X-ray radiation is attenuated instead of being completely blocked (see, e.g., the article by S. Schafer et al., “Filtered region of interest cone-beam rotational angiography,” Medical Physics, Volume 37, No. 2, pages 694 to 703, 2010). Here also, there are disadvantages, however, since additional hardware is used and additional ionizing radiation is used as compared with a fully collimated scan of just the target area (e.g., the VOI). With easily implemented embodiments, only defined sizes of the target area are possible since when traditional collimator leaves are used, overlapping parts would result in increased filtration.

In order to avoid the disadvantages of the previously cited approaches, in DE 10 2012 205 245 A1, an angiographic method for examination of a patient for 3D rotation angiography, in which the opening of the collimator is adjusted during the scan process using a control device in order to record a large volume of the examination object at a large aperture of the collimator and a small volume of the examination object at a small aperture of the collimator, is provided. Therefore, the collimation is continually adjusted during a CT acquisition in order to obtain a densely occupied set of projection images that image the target area in a truncated manner and to obtain a thinly occupied set of non-truncated projection images that completely image the examination object (e.g., therefore, the patient).

Reconstruction approaches that may use such projection images, of which a first part shows only the target area, and another region shows the examination object non-truncated, are known. The subsequently published DE 10 2014 210 420.3 thus concerns a method for reducing truncation artifacts and an X-ray system with which an item of delimitation information describing the delimitation of the patient at least in the truncation portion is used for the determination of estimation data for truncated regions. It is not proposed therein to use projection images accordingly divided into a first part and a second part with dynamically alternating collimation, but rather from the second part of the projection images to derive the delimitation information used therein. For projection image data that includes a first truncated part of projection images and a second non-truncated part of projection images, the method described above in the article by D. Kolditz, in which rather than the pre-CT scan, a forward projection from a three-dimensional image dataset reconstructed from the second part of the projection images takes place, may be used.

Herein, however the problem arises that a practical implementation of the teaching of DE 10 2012 205 245 A1 is extremely difficult from the technical standpoint since the corresponding X-ray system uses a collimator that adjusts itself dynamically during the entire acquisition.

US 2003/0 076 920 A1 discloses an X-ray CT device with a first data detection system and a second data detection system that each have an X-ray tube and an X-ray detector. A reconstruction unit reconstructs image data based on data detected with at least one of the data detection systems. In an exemplary embodiment, both the data detection systems are used in order to record projection images twice, once with a low dose non-truncated and a second time with a higher dose strongly truncated. The data of the non-truncated projection image is to be used to enhance the truncated projection image.

US 2012/0 085 934 A1 relates to a position-determining system for determining a position of an object. A first position-detecting unit detects a first position of the object based on radiation, and a second detection unit detects a second position based on an acceleration of the object and the determined first position. The two position-determining possibilities complement one another.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a simply realized possibility for obtaining, within an imaging process, truncated and non-truncated projection images of an examination object is provided.

In one embodiment of a method, projection images are recorded with an X-ray system that includes at least two recording arrangements (e.g., at least two independently movable recording arrangements), scanning in coordinate systems that are registered or registerable with one another. The recording arrangements include an X-ray radiator with at least one collimator assigned thereto and an X-ray detector. A first part of the X-ray images is recorded with a first of the recording arrangements, and a second part of the X-ray images is recorded with the second of the recording arrangements. In each case, the collimation remains constant during the recording process. The second part of the projection images includes fewer projection images than the first part.

According to one or more of the present embodiments, therefore, a recording scheme that uses a plurality of recording arrangements (e.g., independently movable recording arrangements) is provided. The projection images of the first part (e.g., the truncated projection images) are recorded with a first of the recording arrangements. The collimation is set continually on the target area. The second part of the projection images is recorded with the second recording arrangement. The collimator is set such that the examination object that is recorded (e.g., at least in the plane of the recording trajectory completely) is non-truncated. For example, in one embodiment, the second part of the projection images is recorded completely non-collimated. The projection images of the first part may form a densely occupied projection image set (e.g., relative to the set of the projection directions from which projection images are recorded overall), and the projection images of the second part may form a thinly occupied projection image set. It is therefore provided that the second part of the projection images includes fewer projection images than the first part of the projection images; generally however, the second part of the projection images may include at least two projection images recorded in different projection directions (e.g., at least two projection images lying substantially perpendicularly to one another). From this, improved three-dimensional information may then be derived.

One or more of the present embodiments thus permit the recording of a densely occupied set of truncated projection images of the target area and of a thinly occupied set of non-truncated projection images during a single recording process without a dynamically moving collimator being necessary during the acquisition. This uses no additional hardware and may be realized in a simple manner, for example, on a biplane X-ray system. There are therefore significant advantages in relation to DE 10 2012 205 245 A1.

Herein, the overall dose of X-ray radiation to which the examination object is exposed during the proposed acquisition scheme is comparable with the acquisition scheme described in DE 10 2012 205 245 A1. The additional dose, compared with a conventional scan truncated, in principle, to the target area is herein kept extremely low and may readily be justified in that the projection images of the second part lead to a substantially improved reconstruction quality in the target area as compared with a conventional target area scan.

In this context, in the projection directions in which a projection image of the second part of the projection images is recorded with the second recording arrangement, the recording of a projection image with the first recording arrangement may be omitted. The non-truncated projection image also includes the projection image data of the target area in this projection direction, so that this projection image data does not need to be recorded anew. In this way, further dosage is spared and the same X-ray dose is provided as in the method described according to DE 10 2012 205 245 A1. The second part of the projection images may be used in the reconstruction since for these projection images, no missing projection image data must be estimated.

The coordinate systems of the recording arrangement are registered with one another or, due to known relative settings in the X-ray system, are registerable with one another. This provides that the recording geometries of the two recording arrangements are intrinsically and extrinsically calibrated. Extrinsically calibrated provides that the relative position/orientation of one recording arrangement relative to the other recording arrangement is known in the X-ray system. This has previously been proposed, for example, in known biplane X-ray systems and therefore need not be described in detail here.

The recording arrangements may be moved at least temporarily simultaneously during the joint recording process of the first projection images and the second part of the projection images. This provides that, for example, with circular-type recording trajectories, the two recording arrangements are simultaneously rotated during the recording process. A relative angle between the two recording arrangements may herein be selected as desired. Then, for example, as previously mentioned, imaging with the first recording arrangement may be omitted if the second recording arrangement records a projection image in this projection direction.

One embodiment of the method may be implemented if, as the X-ray system, a biplane X-ray system is used with two C-arms on each of which a recording arrangement is provided. Each of the C-arms is then assigned to a part of the projection images that are to be recorded with the associated recording arrangement, and the assigned collimators are adjusted accordingly. The C-arms may each be fastened to a dedicated holding apparatus (e.g., one C-arm is ceiling mounted, and the other C-arm is floor mounted). A simultaneous movement of the C-arms, and therefore recording arrangements, may be provided. Such a system, however, offers great flexibility since with a corresponding control system of the X-ray system, both an independent movability and also a coupled movability may be achieved.

One or more of the present embodiments may also be used in the field of CT. As the X-ray system, a computed tomography system with two X-ray radiators may thus be used. Such computed tomography systems that are also designated dual-source CT systems therefore possess at least two, possibly separately movable X-ray radiators guided in the gantry. While methods with a dynamic adjustment of the collimator (e.g., the aperture) may not be used in a conventional computed tomography system, since the required speeds may not be achieved at the collimator, the method of one or more of the present embodiments also enables, for the first time in the application in dual source CT, the recording of second parts of the projection images, of which one shows the object to be recorded non-truncated and one shows the object truncated.

In order to determine the truncation information and for artifact-reduced reconstruction of the X-ray image dataset (e.g., of a three-dimensional X-ray image dataset) from two-dimensional projection images, the reconstruction algorithms already known from the prior art may also be used in the context of the present embodiments. For example, the already known methods in the article by D. Kolditz et al. and from DE 10 2014 210 420.3 suggest themselves. Therefore, a configuration of the present embodiments provides that as truncation information, an item of delimitation information of the examination object is determined from a reconstruction of the second part of the projection images. The delimitation information is used in an extrapolation of projection image data lacking in the projection images of the first part due to the collimation onto the target area. However, precisely when a larger number of projection images is present in the second part of the projection images, a three-dimensional intermediate image dataset may be reconstructed from the projection images of the second part in order, by forward projection into the projection directions of the projection images of the first part, to be better able to estimate lacking projection image data. Further reconstruction algorithms taking account of mixed sets of truncated and non-truncated projection images may also be used in the context of the present embodiments.

Summarizing, the present embodiments therefore permit the recording of projection image data that enables high-quality reconstructions of a clinically relevant target area (-volume of interest (VOI)) with extremely low X-ray doses. In order to enable a simple realization of the recording without additional hardware, the advantages of a biplane X-ray system are utilized. Both recording arrangements may be moved (e.g., rotated) simultaneously during the projection image recording. Herein, no dynamically moved collimator is needed.

In addition to the method, the present embodiments also relate to an X-ray system (e.g., a biplane X-ray system) including at least two recording arrangements (e.g., at least two independently movable recording arrangements) scanning in coordinate systems registered or registerable with one another. The recording arrangements include an X-ray radiator with a collimator assigned thereto and an X-ray detector and a control system configured for carrying out the method. All the embodiments relating to the method according to the present embodiments may be transferred similarly to the X-ray system with which the above mentioned advantages may therefore also be achieved. The control system is therefore configured, for example, to control the collimators and the recording arrangement and corresponding associated movement devices such that one of the recording arrangements records truncated projection images of only the target area and the other records non-truncated projection images of the examination object with the target area (e.g., significantly less often). In one embodiment, the control system also includes a truncation information determining unit and a reconstruction unit.

The biplane X-ray system may include two C-arms on which the respective recording arrangements are arranged.

The X-ray system may alternatively be configured, as described, as a dual source computed tomography system. Each of the at least two X-ray radiators is then accordingly assigned a collimator or an aperture that is, for example, controlled accordingly such that by one X-ray radiator, a non-truncated recording and, by the other X-ray radiator, a truncated recording takes place.

### BRIEF DESCRIPTION OF THE DRAWINGS

FIG. 1 shows one embodiment of a biplane X-ray system;

FIG. 2 shows the X-ray system of FIG. 1 in another view;

FIG. 3 is a flow diagram for carrying out one embodiment of a method;

FIG. 4 is an exemplary representation of recording arrangements and corresponding activity in first projection directions; and

FIG. 5 is an exemplary representation of recording arrangements and corresponding activity in second projection directions.

### DETAILED DESCRIPTION

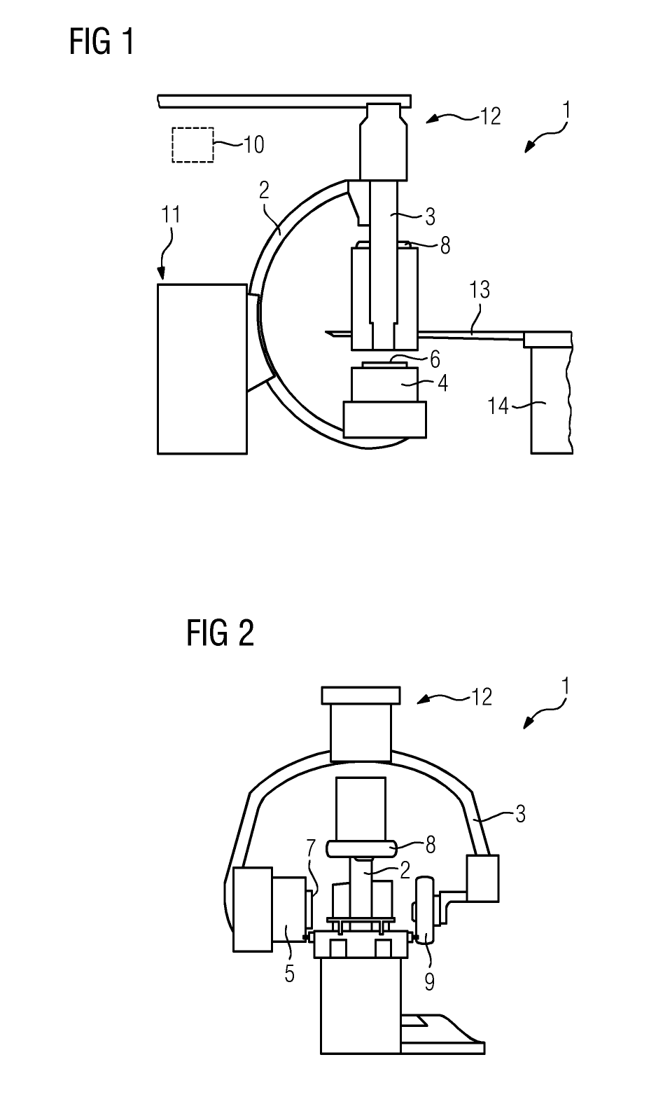

FIGS. 1 and 2 show one embodiment of a biplane X-ray system 1 in two different views. The biplane X-ray system 1 includes two C-arms 2, 3 on each of which, respectively, mutually opposed X-ray radiators 4, 5 with collimators 6, 7 assigned thereto and X-ray detectors 8, 9 are arranged. The X-ray radiators 4 and the X-ray detectors 8 form a first recording arrangement, and the X-ray radiator 5 and the X-ray detector 9 form a second recording arrangement. Coordinate systems of the recording arrangements are, as already proposed for such biplane X-ray systems 1, registered with one another at each time point, since the relative positions of the C-arms 2, 3 and thus of the recording arrangements relative to one another are always known to a control system 10 (only indicated schematically in FIG. 1) of the X-ray system 1, which provides that both an intrinsic and also an extrinsic calibration are provided.

The C-arm 2 is floor-mounted via a holding apparatus 11 with movement devices enabling the movability of the C-arm 2, and the C-arm 3 is ceiling mounted via a corresponding holding apparatus 12.

An examination object (e.g., a patient) may be positioned on a patient table 13 that is supported by a stand 14 and extends freely between the recording arrangements to provide a greatest possible flexibility.

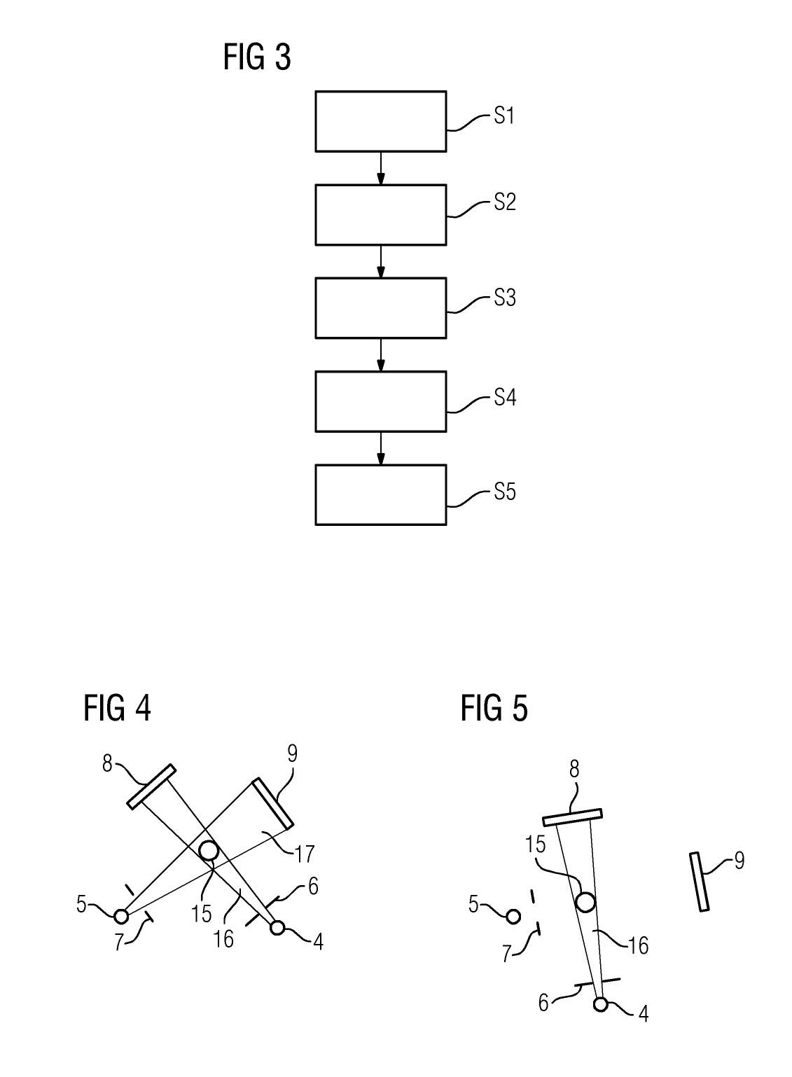

The control system 10 is configured to perform a method of one or more of the present embodiments, which is described in greater detail by reference to FIG. 3. The control system 10 includes a corresponding control system for controlling the recording arrangements (e.g., including the collimators 6, 7) and the movement devices of the holding apparatuses 11, 12, such that in a single recording process, truncated projection images may be recorded as a first part of the projection images and non-truncated projection images may be recorded as a second part of the projection images. The control system 10 includes a truncation information determining unit and a reconstruction unit. The truncation information determining unit is configured to determine from the second part of the projection images an item of truncation information that is used by the reconstruction unit in the reconstruction of a three-dimensional X-ray image dataset from the projection images to reduce truncation artifacts.

FIG. 3 shows an exemplary embodiment of a method. In act S1 before the start of the recording of the projection images, the collimators 6, 7 are adjusted. In the present case, the collimator 6 of the first recording arrangement is adjusted, such that, therefore, with the first recording arrangement, projection images that are collimated onto the target area in the patient and thus truncated may be recorded. However, no collimation is set for the collimator 7.

In act S2, the recording arrangements and thus the C-arms 2, 3 are rotated together and simultaneously around the patient as the examination object to realize a recording trajectory. At specified time points at which particular projection directions are realized through the recording arrangements, respectively, projection images are recorded by the first recording arrangement and the second recording arrangement. Due to the unchanged setting of the collimators 6, 7 during the recording process, the first recording arrangement records only projection images truncated onto the target area (e.g., the first part of the projection images), whereas the second recording arrangement records only non-truncated projection images (e.g., the second part of the projection images). Significantly fewer projection images of the second part than projection images of the first part are recorded, which provides that while the first part of the projection images is a densely occupied set of projection images, the second part of the projection images is a thinly occupied set of projection images that, however, contains at least two projection images in different projection directions in order to be able to derive three-dimensional truncation information. The recording of a projection image by the first recording arrangement is suppressed if at this point a projection image of the second part of the projection images was recorded or will be recorded in order to reduce the radiation load further.

FIG. 4 shows, by way of example, a first position of the recording arrangements. For the sake of clarity, the target area 15 is also shown. As is apparent, the collimator 6 is so narrowly set that through the associated radiation field 16 (e.g., indicating the activity of the recording arrangement), it is not the whole examination object (e.g., the patient) that is detected, but only the target area 15. In the position shown, as is apparent from the radiation field 17, the second recording arrangement is also active, the collimator 7 of which is adjusted so that no collimation is carried out. This provides that the whole examination object is recorded non-truncated in this projection direction.

In contrast thereto, in the second position of the recording arrangements according to FIG. 5, only the radiation field 16 of the first X-ray radiator 4 is visible; thus, only the first recording arrangement is active. In the projection direction that corresponds to the position of the second recording arrangement, therefore, no projection image of the second part of the projection images should be recorded. When the first recording arrangement reaches the position, during rotation, in which the second recording arrangement is situated in the representation of FIG. 4, no projection image of the first part is recorded, since for this, a projection image of the second part already exists. Returning to FIG. 3, in act S3, an item of truncation information is determined from the second part of the projection images. Therein, for example, for an extremely thin scan, an item of delimitation information of the examination object may be obtained as truncation information that the subsequent extrapolation in act S4 of the projection image data of the first part of the projection images lacking due to the truncation co-determines. However, with a larger number of projection images in the second part of the projection images, an intermediate image dataset may be reconstructed in order, by forward projection in act S4, to complement lacking projection image data in the projection images of the first part of the projection images.

Other approaches may also be provided; the invention is thus not restricted to the truncation artifact-reducing reconstruction methods described.

Once lacking projection image data for all the projection images has been complemented, all the projection images (e.g., the projection images of the second part and the complemented images of the first part) may be used in act S5 to reconstruct the three-dimensional X-ray image dataset that shows the target area 15 artifact-reduced and in high quality.

Although the invention has been illustrated and described in detail based on the exemplary embodiments, the invention is not restricted by the examples given. Other variations may be derived therefrom by a person skilled in the art without departing from the protective scope of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for determining an X-ray image dataset of a target area of an examination object, the method comprising:
recording, by an X-ray system, projection images showing the target area from different projection directions along a recording trajectory; and
reconstructing, from the projection images, a higher-dimensional X-ray image dataset, a first part of the projection images being recorded collimated onto the target region and a second part of the projection images showing the examination object non-truncated in at least one direction, so that from the second part of the projection images at least one item of truncation information that is used in the reconstruction of the X-ray image dataset for reducing truncation artifacts is derived,
wherein the projection images are recorded with an X-ray system, the X-ray system comprising at least two recording arrangements, scanning in coordinate systems that are registered or registerable with one another, the at least two recording arrangements comprising an X-ray radiator with at least one collimator assigned to the X-ray radiator, and an X-ray detector,
wherein the first part of the X-ray images are recorded with a first of the at least two recording arrangements, and the second part of the X-ray images is recorded with a second of the at least two recording arrangements, in each case, the collimation remaining constant during the recording process, and
wherein the second part of the projection images comprises fewer projection images than the first part of the projection images.

2. The method of claim 1, wherein the second part of the projection images are recorded completely non-collimated, the second part of the projection images comprises at least two projection images recorded in different projection directions, or a combination thereof.

3. The method of claim 1, wherein in the projection directions in which a projection image of the second part of the projection images is recorded with the second recording arrangement, the recording of a projection image with the first recording arrangement is omitted.

4. The method of claim 1, wherein the at least two recording arrangements are moved at least temporarily simultaneously during a joint recording process of the first part of the projection images and the second part of the projection images.

5. The method of claim 1, wherein the X-ray system comprises a biplane X-ray system, the biplane X-ray system comprising two C-arms, a recording arrangement being provided on each of the two C-arms.

6. The method of claim 5, wherein the two C-arms are each fastened to a dedicated holding apparatus.

7. The method of claim 6, wherein one of the two C-arms is ceiling mounted, and the other of the two C-arms is floor mounted.

8. The method of claim 1, wherein the X-ray system comprises a computed tomography system with two X-ray radiators.

9. The method of claim 1, wherein as the item truncation information, an item of delimitation information of the examination object is determined from a reconstruction of the second part of the projection images, the item of delimitation information being used in an extrapolation of projection image data lacking in the projection images of the first part due to the collimation onto the target area.

10. An X-ray system comprising:
at least two recording arrangements scanning in coordinate systems registered or registerable with one another, the recording arrangements comprising:
an X-ray radiator with a collimator assigned to the X-ray radiation;
an X-ray detector; and
a control system configured to:
record projection images showing the target area from different projection directions along a recording trajectory; and
reconstruct, from the projection images, a higher-dimensional X-ray image dataset, a first part of the projection images being recorded collimated onto the target region and a second part of the projection images showing the examination object non-truncated in at least one direction, so that from the second part of the projection images at least one item of truncation information that is used in the reconstruction of the X-ray image dataset for reducing truncation artifacts is derived,
wherein the first part of the X-ray images are recorded with a first of the at least two recording arrangements, and the second part of the X-ray images is recorded with a second of the at least two recording arrangements, in each case, the collimation remaining constant during the recording process, and
wherein the second part of the projection images comprises fewer projection images than the first part of the projection images.

11. The X-ray system of claim 10, wherein the X-ray system is a biplane X-ray system.

12. The X-ray system of claim 10, wherein the second part of the projection images are recorded completely non-collimated, the second part of the projection images comprises at least two projection images recorded in different projection directions, or a combination thereof.

13. The X-ray system of claim 10, wherein in the projection directions in which a projection image of the second part of the projection images is recorded with the second recording arrangement, the recording of a projection image with the first recording arrangement is omitted.

14. The X-ray system of claim 10, wherein the at least two recording arrangements are configured to move at least temporarily simultaneously during a joint recording process of the first part of the projection images and the second part of the projection images.

15. The X-ray system of claim 10, wherein the X-ray system comprises a biplane X-ray system, the biplane X-ray system comprising two C-arms, a recording arrangement being provided on each of the two C-arms.

16. The X-ray system of claim 15, wherein the two C-arms are each fastened to a dedicated holding apparatus.

17. The X-ray system of claim 16, wherein one of the two C-arms is ceiling mounted, and the other of the two C-arms is floor mounted
